(19) United States
(12) Patent Application Publication
(10) Pub. No.: US 2025/0050050 A1
(43) Pub. Date: Feb. 13, 2025

(54) OXYGEN PROVIDING MICROPHONE STAND

(71) Applicant: Patrick McAllister, Conifer, CO (US)

(72) Inventor: Patrick McAllister, Conifer, CO (US)

(21) Appl. No.: 18/448,460

(22) Filed: Aug. 11, 2023

Related U.S. Application Data
(60) Provisional application No. 63/462,338, filed on Apr. 27, 2023.

(51) Int. Cl. A61M 16/10; H04R 1/02

(52) U.S. Cl. CPC A61M 16/1005; H04R 1/028; A61M 2209/084

(57) ABSTRACT

Aspects of oxygen providing microphone stands are described. The oxygen providing microphone stand can include an oxygen providing component that connects to a microphone stand. The oxygen providing component outputs oxygen proximal to a microphone connected to the microphone stand. The oxygen providing microphone stand can include an oxygen delivery pathway that connects from the oxygen providing component to the input oxygen connector.

### CROSS REFERENCE TO RELATED APPLICATIONS

This application claims priority to and the benefit of U.S. Provisional Application No. 63/462,338, filed on Apr. 27, 2023 and entitled “OXYGEN PROVIDING MICROPHONE STAND, which is hereby incorporated herein by reference in its entirety.

### BACKGROUND

Microphone stands can include a base, a shaft and a boom arm so that an object such as a microphone or sheet music can be supported using a device attached to the microphone stand. This allows, for example, a singer or performer to articulate the stand to place a microphone (or sheet music, etc.) directly in front of the mouth to maximize effect of the microphone. The shaft and boom can include a number of articulation points such as telescoping tubes, pivot joints, and the like.

Singers, speakers, and other performers can use the voice and breath extensively while performing. For example, a singer can sing notes, a speaker can use a powerful tone, or a wind instrument player can blow heavily for long durations. This can take a toll on any performance. Even if the microphone stand is properly positioned to maximize audio quality and pickup, the performance can suffer if the performer becomes winded during the performance piece. As a result, there is a need for devices that can improve vocal and instrument performances in association with a microphone stand.

### BRIEF DESCRIPTION OF THE DRAWINGS

Aspects of the present disclosure will be more readily appreciated upon review of the detailed description of its various embodiments, described below, when taken in conjunction with the accompanying drawings depicted in the Figures.

FIG. 1 shows an example of an oxygen providing microphone stand according to various embodiments of the present disclosure.

FIG. 2 shows another example of an oxygen providing microphone stand according to various embodiments of the present disclosure.

FIG. 3 shows another example of an oxygen providing microphone stand provided by affixing an oxygen providing microphone stand kit to a microphone stand according to various embodiments of the present disclosure.

FIGS. 4A and 4B show examples of output oxygen components for oxygen providing microphone stands according to various embodiments of the present disclosure.

FIG. 5 shows an example of a pivot joint for oxygen providing microphone stands according to various embodiments of the present disclosure.

FIG. 6 shows an example of a base portion of an oxygen providing microphone stand that includes a coiled air hose according to various embodiments of the present disclosure.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

The oxygen providing microphone stand can help singers and speakers increase their energy and performance as they breathe enhanced oxygen that is enhanced by dispersing or otherwise providing oxygen from an oxygen source. An oxygen source can include an oxygen canister that connects via a connector at the base, travels up the stand and boom, while gently diffusing a cloud of oxygen that is most concentrated within several inches of the microphone. This technology can help singers and speakers maintain healthy blood-oxygen levels for a longer and more powerful and dynamic performance.

Example embodiments are shown and described in the figures. Generally, the device can include a microphone stand, at least one oxygen supply fitting, at least one oxygen supply line, and an oxygen providing component that disperses oxygen proximal to the microphone connection component of the microphone stand. In some examples, an oxygen hose compression fitting is located at the base of the stand, allowing any oxygen concentrator to interface. In some examples, the microphone stand has an oxygen tank built into the base. The oxygen providing component can include a small horn-lens or other diffuser component integrated or connected beneath or otherwise proximal to the microphone, dispersing oxygen in a unidirectional degree pattern or another pattern proximal to a direction of a user of the microphone. The oxygen providing component can have several small holes to disperse the oxygen. Oxygen flow can be manually or automatically regulated and adjusted using a flow adjustment component proximal to the oxygen providing component.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. While features can be shown and described with respect to a particular figure, the features in one figure are applicable to the other figures of the disclosure. Any recited method can be carried out in the order of events recited or in any other order that is logically possible.

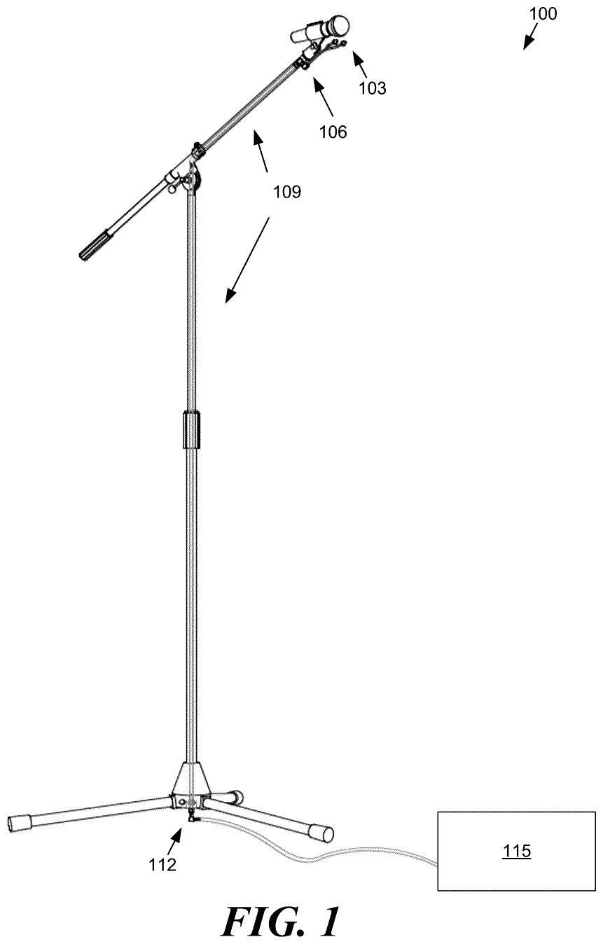

Moving to FIG. 1, shown is an oxygen providing microphone stand 100. The concepts and features described with respect to the oxygen providing microphone stand 100 in FIG. 1 can be combined with those of the other figures herein. The oxygen providing microphone stand 100 can include an oxygen providing component 103, an output oxygen tube fitting or connector 106, an oxygen delivery pathway 109, and an input oxygen tube fitting or connector 112. In some examples, the oxygen source 115 can be considered part of an oxygen providing microphone stand system, and in other examples, it can be considered a separate component from the system.

The oxygen providing component 103 can provide output oxygen proximal to the microphone (or other object) holder at the end of the oxygen providing microphone stand 100. The oxygen providing component 103 can include a flexible tube, a rigid tube, or another type of oxygen tube. In some examples, the oxygen providing component 103 can include a horn shaped tip, a perforated tip, or another type of air dispersion tip that disperses air. In some examples, the oxygen providing component 103 can include a tip or another component that provides a wide air dispersion that provides a softer flow over a larger zone of air. In some examples, the oxygen providing component 103 can include a tip or another component that provides a narrow air dispersion that provides a more directed flow to a smaller zone in a particular direction. The tip can be adjustable to adjust a dispersion, from a wide air dispersion that provides a softer flow over a larger zone of air, to a narrow air dispersion that provides a more directed flow to a smaller zone in a particular direction. In some examples, the oxygen providing component 103 can also include an articulation point at a base, tip, or a midpoint that allows the direction of the air flow to be adjusted, whether a wide or narrow dispersion is provided.

The position of the oxygen providing component 103 enables adjustment of the articulation points of the microphone stand to adjust the output oxygen position of the oxygen providing component 103. The oxygen providing component 103 is adjustable, and can be adjusted such that the output position of oxygen flow from the oxygen providing component 103 points towards a microphone audio pickup position where the microphone detects a higher portion of audio than other positions relative to the microphone. The oxygen providing component 103 can also be adjusted so that the oxygen flow points towards a preferred position of a face of a singer, speaker, musician, or other performer, since some performers do not utilize an optimal microphone audio pickup position. In any case, as the articulation points of the oxygen providing microphone stand 100 are adjusted, the output position of oxygen flow from the oxygen providing component 103 is also moved as a result.

The output oxygen connector 106 can connect the oxygen providing component 103 (or a tube connected thereto) to an interior space or cavity inside the oxygen providing microphone stand 100. The output oxygen connector 106 can include one or more slip-on, push-on, threaded, or other type of fitting or adapter that connects to the oxygen providing component 103. The adapter can include a male adapter or a female adapter. Another adapter can connect to a tube in the interior space or cavity inside the oxygen providing microphone stand 100, which can also include any number of fitting types. The adapters of the output oxygen connector 106 and any connection between them can be of one or more of: brass, aluminum, steel, or other metals, plastic, and other materials.

The output oxygen connector 106 can include a 90 degree or another angled connection so that the output oxygen connector 106 connects to the oxygen providing component 103 in a direction parallel to the microphone and/or a boom of the oxygen providing microphone stand 100. However, in other examples, the output oxygen connector 106 can be a straight connector that is perpendicular to the boom of the oxygen providing microphone stand 100. For example, if the microphone stand does not have a boom, a straight connector can be used, since the microphone is not in line with the vertical portion of the oxygen providing microphone stand 100. In some examples, the output oxygen connector 106 can also include a second adapter that connects to a tube inside the oxygen providing microphone stand 100. This can be considered an internal adapter while the adapter that connects to the oxygen providing component 103 can be considered an external adapter of the output oxygen connector 106. However, in further examples, no internal adapter is used. For example, if the oxygen delivery pathway 109 is formed by one or more air tight internal area of the oxygen providing microphone stand 100 to which the output oxygen connector 106 connects. The oxygen delivery pathway 109 shown is internal to the oxygen providing microphone stand 100, and can be referred to as an internal oxygen delivery pathway 109. However, in some cases the oxygen delivery pathway 109 can be wholly or partially exterior to the rigid portions of the oxygen providing microphone stand 100. The air tight internal area can be formed by the poles, extensions, conduits, or other rigid structural components of the oxygen providing microphone stand 100, which can be lined using a coating or can be formed from or connected to air tight material internal to the rigid structural components. The output oxygen connector 106 can also connect securely to a wall of a tube of the boom or vertical extension of the oxygen providing microphone stand 100.

The oxygen delivery pathway 109 can be provided using one or more internal oxygen tubes inside a cavity provided by the boom and vertical oxygen providing microphone stand 100. For example, a single continuous tube can be used from the output oxygen connector 106 to the input oxygen connector 112, or multiple different tubes can be connected together internally. The tubes can be connected together using an adapter that in various examples can be connected rigidly to a portion of the interior of the oxygen providing microphone stand 100, or can be unconnected to the oxygen providing microphone stand 100. A specialized tube-path-providing pivot joint can be provided that enables an air tube to pass through the pivot joint without being crimped or cut as the oxygen providing microphone stand 100 and its boom are adjusted at the tube-path-providing pivot joint. The path can remain open even when the joint is tightened down and locked in place.

The oxygen delivery pathway 109 can also be provided using one or more air-tight internal area of the oxygen providing microphone stand 100. In some examples, a single internal area can be provided that enables air-tight operation of all of the articulation points, including extension points angle adjustments, and other articulations between the output oxygen connector 106 and the input oxygen connector 112. In further examples, one or more interior areas or zones of the oxygen providing microphone stand 100 can be air-tight and can be used as a part of the oxygen delivery pathway 109. For example, a boom or a portion of the boom can be air tight in lieu of an internal tube in that portion. A vertical (or other) pole or a portion of that pole can be air tight in lieu of an internal tube in that portion.

The input oxygen connector 112 can be connected to a source oxygen tube that connects between the oxygen providing microphone stand 100 and an oxygen source 115. The input oxygen connector 112 can include one or more slip-on, push-on, threaded, or other type of fitting or adapter that connects to the source oxygen tube or the oxygen source 115 directly. The adapter can include a male adapter or a female adapter. Another adapter can connect to a tube in the interior space or cavity inside the oxygen providing microphone stand 100, which can also include any number of fitting types. The adapters of the input oxygen connector 112 and any connection between them can be of one or more of: brass, aluminum, steel, or other metals, plastic, and other materials.

The input oxygen connector 112 can include a 90 degree or another angled connection. However, in other examples, the input oxygen connector 112 can be a straight connector that is parallel or in line with a vertical extension of the oxygen providing microphone stand 100. For example, if the microphone stand does not have a boom, a straight connector can be used, since the microphone is not in line with the vertical portion of the oxygen providing microphone stand 100. In some examples, the input oxygen connector 112 can also include a second adapter that connects to a tube inside the oxygen providing microphone stand 100. This can be considered an internal adapter while the adapter that connects to the oxygen providing component 103 can be considered an external adapter of the input oxygen connector 112. However, in further examples, no internal adapter is used. For example, if the oxygen delivery pathway 109 is formed by one or more air tight internal area of the oxygen providing microphone stand 100 to which the input oxygen connector 112 connects. The input oxygen connector 112 can also connect securely to a wall of a tube of the vertical pole or portion of the oxygen providing microphone stand 100 from which the input oxygen connector 112. The input oxygen connector 112 is shown under the stand within a footprint of a base of the stand. However, in other examples where there is no space under a base or foot of the stand, the input oxygen connector 112 can be connected to a top or side of the base, to a vertical pole or other pole or portion of the oxygen providing microphone stand 100.

The oxygen source 115 can include an oxygen concentrator device, an oxygen tank, or another type of oxygen source. Oxygen concentrators can include large devices and portable devices that can filter and concentrate the surrounding air, generating medical-grade oxygen. Oxygen tanks can include large or portable compressed oxygen tanks, oxygen tanks with liquid oxygen or compressed gaseous oxygen, and so on.

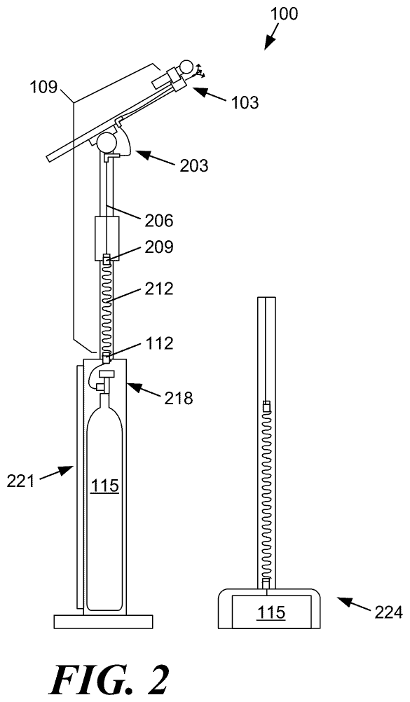

FIG. 2 shows another example of an oxygen providing microphone stand 100. The concepts and features described with respect to the oxygen providing microphone stand 100 in FIG. 2 can be combined with those of the other figures herein. The oxygen providing microphone stand 100 in FIG. 2 can include an oxygen providing component 103, an oxygen delivery pathway 109, and an oxygen source 115.

In this example, the oxygen providing component 103 can include a nozzle, tube, tip, or opening that is integrated with the boom (or other pole or extension) of the oxygen providing microphone stand 100. Since the microphone holder of the oxygen providing microphone stand 100 can connect to an end of the boom (or other pole or extension), an integrated oxygen providing component 103 at a distal end of the boom or extension can output the flow of oxygen, rather than having a separate oxygen providing component 103 connected to that boom or extension.

In some examples, the oxygen providing component 103 can include a tip or another component that provides a wide air dispersion that provides a softer flow over a larger zone of air. In some examples, the oxygen providing component 103 can include a tip or another component that provides a narrow air dispersion that provides a more directed flow to a smaller zone in a particular direction. The tip can be adjustable to adjust a dispersion, from a wide air dispersion that provides a softer flow over a larger zone of air, to a narrow air dispersion that provides a more directed flow to a smaller zone in a particular direction. In some examples, the oxygen providing component 103 can also include an articulation point at a base, tip, or a midpoint that allows the direction of the air flow to be adjusted, whether a wide or narrow dispersion is provided.

The oxygen delivery pathway 109 in this figure is shown to include multiple internal portions that are connected therebetween by an external oxygen pathway 203. While the oxygen delivery pathway 109 is shown as a number of tubes including straight and coil shaped tubes, the oxygen delivery pathway 109 can additionally or alternatively include air tight internal areas formed by the structural components of the oxygen delivery pathway 109 as discussed earlier.

The external oxygen pathway 203, or external portion of the oxygen delivery pathway 109, can include an expandable and collapsible device such as a coiled tube or other device that connects to “external pathway” connectors in order to avoid routing the air through a pivot joint or other articulation point. The collapsible device can include a continuous ridge or multiple ridges that enable expansion and contraction as well as articulation of at least a portion of the external oxygen pathway 203. The two “external pathway” connectors can connect to the internal portions of the oxygen delivery pathway 109 on each side of the pivot joint or other articulation point. The “external pathway” connectors can include one or more slip-on, push-on, threaded, or other type of fitting or adapter that connects to the oxygen providing component 103. The adapter can include a male adapter or a female adapter. Another adapter can connect to a tube in the interior space or cavity inside the oxygen providing microphone stand 100, which can also include any number of fitting types. The adapters any connection between them can be of metal such as brass, aluminum, steel, or other metals, plastic, and other materials appropriate for moving air.

The external oxygen pathway 203 can connect to a tube 206 or other internal portion of the oxygen delivery pathway 109 that is in the vertical support extension as shown. In this example, the tube 206 is connected, via an internal adapter 209 that connects to a coil-shaped tube 212 inside the oxygen providing microphone stand 100. The coil-shaped tube 212 can enable the oxygen providing microphone stand 100 to be articulated, for example by adjusting the vertical extension so that the oxygen providing microphone stand 100 is taller or shorter. If the oxygen providing microphone stand 100 is adjusted to be taller (or otherwise longer in total length), then the coil-shaped tube 212 can expand without putting stress on or breaking the oxygen delivery pathway 109. If the oxygen providing microphone stand 100 is adjusted to be shorter (or otherwise shorter or smaller in total length), then the coil-shaped tube 212 can contract. In an instance in which the upper tube 206 extends through a tube-path-providing pivot joint rather than using the external oxygen pathway 203, the upper tube 206 can be a straight upper tube that enables it to slide through the tube-path-providing pivot joint as the microphone stand 100 is adjusted to be shorter or longer in total internal length along the oxygen delivery pathway 109.

The input oxygen connector 112 can be connected to a source oxygen tube that connects between the oxygen providing microphone stand 100 and an oxygen source 115. The input oxygen connector 112 can include one or more slip-on, push-on, threaded, or other type of fitting or adapter that connects to the source oxygen tube or the oxygen source 115 directly. The adapter can include a male adapter or a female adapter. Another adapter can connect to a tube in the interior space or cavity inside the oxygen providing microphone stand 100, which can also include any number of fitting types. The adapters of the input oxygen connector 112 and any connection between them can be of one or more of: brass, aluminum, steel, or other metals, plastic, and other materials.

The input oxygen connector 112 can be a straight connector as shown, and can include an internal oxygen connector 112 internal to the oxygen providing microphone stand 100. In this example, the oxygen source 115 can be held within an integrated oxygen source housing 218 of the oxygen providing microphone stand 100. The oxygen source housing 218 can include a hinged or unhinged access point 221 such as a door, flap, or other opening at the side as shown; or a concealed opening point at a bottom of the oxygen providing microphone stand 100 so that the oxygen source 115 can be replaced and/or serviced.

As discussed above, the oxygen source 115 can include an oxygen concentrator device, an oxygen tank, or another type of oxygen source. In some examples where the oxygen source 115 is provided with the oxygen providing microphone stand 100 the oxygen source 115 can be formed in a form factor such as a tall and thin form factor such as a tall and thin oxygen tank as shown, or a tall and thin concentrator device. If a concentrator device is used, in some examples, the oxygen source housing 218 can include vents sufficient to enable air flow to the concentrator. Alternatively, the oxygen source housing 218 can be wide and flatter, and can form a base of the oxygen providing microphone stand 100, and the oxygen source 115 can include a form factor that can fit within the base of the oxygen providing microphone stand 100, as shown in the detail 224.

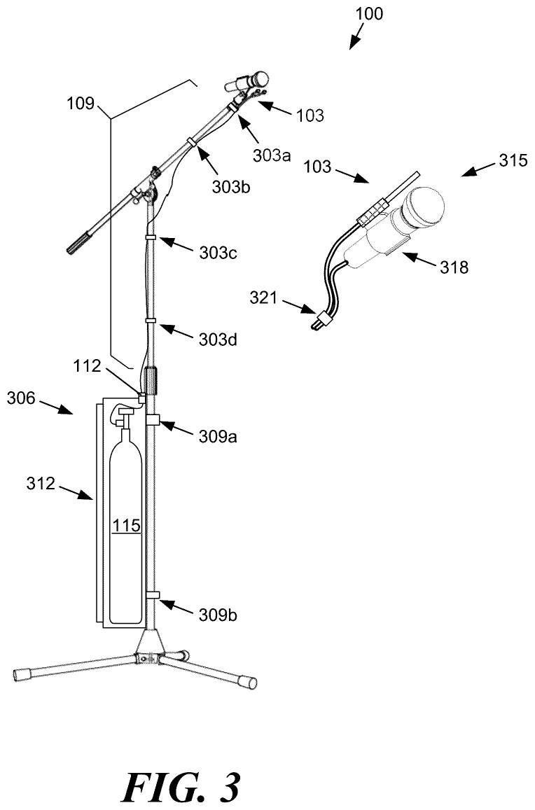

FIG. 3 shows another example of an oxygen providing microphone stand 100 provided by affixing an oxygen providing microphone stand kit to a standard microphone stand according to various embodiments of the present disclosure. The concepts and features described with respect to the oxygen providing microphone stand 100 in FIG. 3 can be combined with those of the other figures herein. The oxygen providing microphone stand kit shown can include an oxygen providing component 103, an external oxygen delivery pathway 109, any number of microphone stand tube connectors 303 (e.g., 303a-303d), and in some examples, an oxygen source housing 306 and any number of microphone stand oxygen source housing connectors 309 (e.g., 309a-303b).

The oxygen providing component 103 can be similar to any of the oxygen providing components 103 and features described with respect to the other figures. The external oxygen delivery pathway 109 can include straight or coiled oxygen tubing. In some examples, the tubing can be black tubing or transparent tubing to reduce visibility of the tubing. In some examples, the tubing can include one or more rigid sections so that the rigid sections can align with the poles, conduits, or elongate structural components of the microphone stand. The external oxygen delivery pathway 109 can include any number of adapters that adapt between flexible and rigid portions of the external oxygen delivery pathway 109.

The microphone stand tube connectors 303a-303d can include detachably attached connectors that have one portion shaped to clip, snap, push-on or otherwise detachably attach or connect to a microphone boom or stand, and another portion shaped to clip, snap, or otherwise permanently (or detachably) connect to an air hose or similarly sized tube or cable. In some examples, the microphone stand tube connectors 303a and 303b can be shaped to slide over and connect to a common diameter of a microphone boom (and an air hose) while the microphone stand tube connectors 303c and 303d can be shaped to slide over and connect to a common diameter of a microphone stand. The microphone stand tube connectors 303a-303d can include connectors that include one or more spring loaded connectors that can be squeezed to open, non-spring connectors, hook and loop type connectors, and so on.

The oxygen source housing 306 can be a housing that includes connection points that connect to the oxygen source housing connectors 309, or includes integrated oxygen source housing connectors 309 that connect to a common diameter of a microphone stand. The oxygen source housing connectors 309 can include detachably attached connectors that have one portion shaped to clip, snap, push-on or otherwise detachably attach or connect to a microphone stand of a common diameter for microphone stands. The oxygen source housing connectors 309 can include a portion that detachably or permanently attaches to the oxygen source housing 306. Some of these connections can include a portion that slides through a hole of the oxygen source housing 306, pushes into a grooved recess of the oxygen source housing 306, or otherwise connects to the oxygen source housing 306. The oxygen source housing 306 can include a foot or rubberized that helps to support the oxygen source housing 306. The foot or rubberized portion can make contact with a floor or other surface that also supports the microphone stand. The oxygen source housing 306 can come in a number of different form factors for different types of oxygen sources 115, as discussed with respect to the other figures for other housings such as integrated housings.

The detail item 315 shows a microphone clip 318 that clips to a microphone rather than to the tube connectors 303a that connects to the microphone boom. The microphone clip 318 can be part of the oxygen providing microphone stand kit that converts a microphone stand into an oxygen providing microphone stand 100. The microphone clip 318 can also connect to the oxygen providing component 103. In some examples, the oxygen providing component 103 can include, incorporate or can be permanently attached to the microphone clip 318 the tube connectors 303a.

The detail item 315 also shows a tube-to-cable connector 321 that connects to a microphone cable and the external oxygen delivery pathway 109. In some examples, the kit can include connects the external oxygen delivery pathway 109 to the microphone cable. In some examples, an oxygen source housing 306 and an oxygen source 115 can be placed proximal to a microphone cable connector, and the kit can be an oxygen providing microphone kit that does not connect to a microphone stand, but rather provides the external oxygen delivery pathway 109 from the microphone clip 318 to the oxygen source 115 by connecting to a microphone cable using multiple tube-to-cable connectors 321.

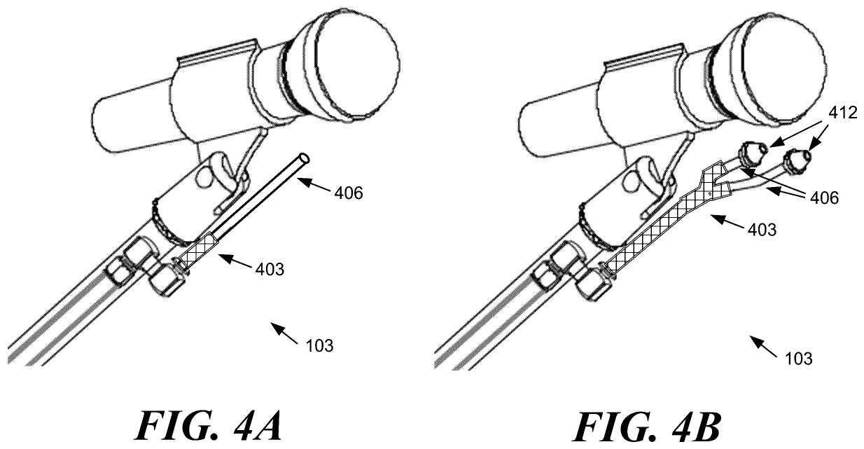

FIG. 4A shows one example of an oxygen providing component 103. In this example, the oxygen providing component 103 includes an adjustable oxygen flow direction component 403, and a rigid tube portion 406. The adjustable oxygen flow direction component 403 can be a component that fits over, connects around, or is incorporated into an oxygen pathway of the oxygen providing component 103. The adjustable oxygen flow direction component 403 can enable the oxygen providing component 103 to be positioned as desired without crimping oxygen pathway of the adjustable oxygen flow direction component 403. While it can be repositioned, the adjustable oxygen flow direction component 403 can hold in position once adjusted. The adjustable oxygen flow direction component 403 can also include a mechanical device that includes an air-tight pathway and one or more hard articulation points, such as a ball joint with an air pathway, a pivot joint with an air pathway, and so on.

FIG. 4B shows another example of an oxygen providing component 103. In this example, the oxygen providing component 103 includes an adjustable oxygen flow direction component 403 that includes a wye or splitter that splits a single oxygen pathway into multiple oxygen pathways. The oxygen providing component 103 of FIG. 4B also small horn-lens, perforated diffuser, or other diffuser components 412 that disperses oxygen in a predetermined pattern proximal to a direction of a user of the microphone. While both diffuser components 412 are pointed for use by a single performer in this example, they can also be pointed in different directions for concurrent use by multiple performers. In some examples, the diffuser devices or components 412 can also include one or more rotating or other adjustment component that can increase or decrease area of dispersion and/or increase or decrease air flow.

As with FIG. 4A, the adjustable oxygen flow direction component 403 of FIG. 4B can also be a component that fits over, connects around, or is incorporated into an oxygen pathway of the oxygen providing component 103. The adjustable oxygen flow direction component 403 can enable the oxygen providing component 103 to be positioned as desired without crimping oxygen pathway of the adjustable oxygen flow direction component 403. While it can be repositioned, the adjustable oxygen flow direction component 403 can hold in position once adjusted. The adjustable oxygen flow direction component 403 can also include a mechanical device that includes an air-tight pathway and one or more hard articulation points, such as a ball joint with an air pathway, a pivot joint with an air pathway, and so on.

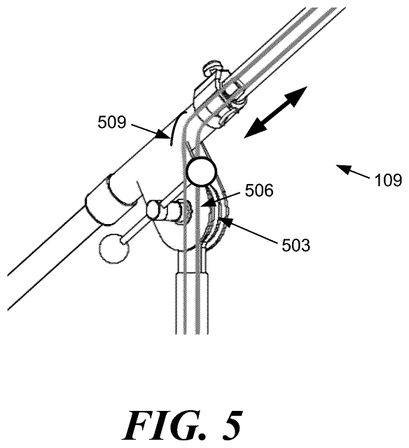

FIG. 5 shows an example of a tube-path-providing pivot joint 503 that includes an oxygen pathway opening 506. This specialized tube-path-providing pivot joint 503 can enable an air tube to pass through the pivot joint without being crimped or cut as the oxygen providing microphone stand 100 and its boom are adjusted at the tube-path-providing pivot joint. The path can remain open even when the tube-path-providing pivot joint 503 is tightened down and locked in place. In some examples, the tube-path-providing pivot joint 503 can include a tube director component 509 that guides the air tubing to pass through the tube-path-providing pivot joint 503 rather than getting caught in the boom portion. In some examples, the boom portion can be fixed so that it does not extend inward and outward. In other examples, the boom portion can include a slot that faces downward towards the vertical extension of the microphone stand, which enables the boom to slide in the directions indicated by the arrows, while also enabling the air tube to exit the boom through the tube-path-providing pivot joint 503. Alternatively, the oxygen pathway opening 506 of the tube-path-providing pivot joint 503 can include an air-tight pathway so that the oxygen delivery pathway 109 remains open and air tight through tightening and loosing and angle adjustments.

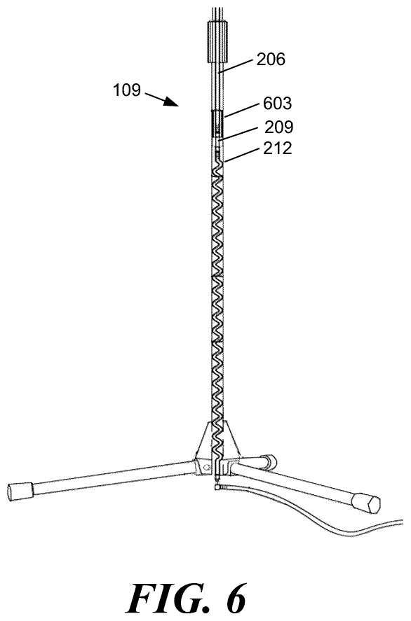

FIG. 6 shows an example of a base portion of an oxygen providing microphone stand 100 with a portion of an oxygen delivery pathway 109 that includes a straight tube 206, an internal adapter 209, a coil-shaped tube 212, and a weight 603. The internal adapter 209 connects the straight tube 206 to the coil-shaped tube 212 inside the oxygen providing microphone stand 100. The weight 603 can ensure that the tube passes through the tube-path-providing pivot joint 503 of FIG. 5, without allowing slack to build up so that the tube will not crimp. In some examples, the weight 603 can connect to, be incorporated with, or otherwise be provided by the internal adapter 209.

It should be emphasized that the above-described embodiments of the present disclosure are merely possible examples of implementations set forth for a clear understanding of the principles of the disclosure. Many variations and modifications can be made to the above-described embodiments without departing substantially from the spirit and principles of the disclosure. While concepts of the present disclosure are discussed with respect to a particular figure, the concepts can also be used in connection with the other figures. All such modifications and variations are intended to be included herein within the scope of this disclosure and protected by the following claims.

What is claimed:

1. An oxygen providing microphone stand comprising:
a microphone stand;
an input oxygen connector connected to the microphone stand;
an oxygen providing component that connects to the microphone stand, and outputs oxygen proximal to a microphone connected to the microphone stand; and
an oxygen delivery pathway that connects from the oxygen providing component to the input oxygen connector.

2. The oxygen providing microphone stand of claim 1, wherein the oxygen delivery pathway is at least partially internal to the microphone stand.

3. The oxygen providing microphone stand of claim 1, wherein the input oxygen connector connects to an oxygen tube that leads to an oxygen source.

4. The oxygen providing microphone stand of claim 1, further comprising an oxygen source housing that is incorporated with or connected to the microphone stand.

5. The oxygen providing microphone stand of claim 1, wherein the oxygen delivery pathway connects to the oxygen providing component using an output oxygen connector, and the output oxygen connector connects between: an internal portion of the oxygen delivery pathway internal to the microphone stand, and the oxygen providing component that is external to the microphone stand.

6. The oxygen providing microphone stand of claim 1, wherein the oxygen providing component is output from a distal end of a boom of the microphone stand.

7. The oxygen providing microphone stand of claim 1, wherein the oxygen providing component is output from a distal end of a boom of the microphone stand.

8. An oxygen providing system that converts a microphone stand to an oxygen providing microphone stand, the system comprising:
an oxygen providing component comprising a clip that is adapted to connect to a microphone stand or a microphone, and outputs oxygen proximal to the microphone; and
an oxygen delivery pathway that connects from the oxygen providing component to an input oxygen connector.

9. The system of claim 8, wherein the oxygen delivery pathway is external to the microphone stand.

10. The system of claim 8, further comprising an oxygen source housing that is detachably attached to the microphone stand using at least one connector.

11. The system of claim 8, wherein the input oxygen connector connects to an oxygen tube that leads to an oxygen source.

12. The system of claim 8, further comprising an oxygen source housing that is connected to the microphone stand using at least one detachable connector.

13. The system of claim 12, wherein the oxygen delivery pathway comprises an input oxygen connector that connects through a wall of the oxygen source housing.

14. The system of claim 8, wherein the oxygen delivery pathway is detachably attached to the microphone stand using a plurality of connectors that are adapted to connect to the oxygen delivery pathway and at least one portion of the microphone stand.

15. An apparatus comprising:
a microphone stand;
an oxygen providing component that connects to the microphone stand, and outputs oxygen proximal to a microphone connected to the microphone stand; and
an oxygen delivery pathway that connects from the oxygen providing component to the input oxygen connector.

16. The apparatus of claim 15, wherein the oxygen delivery pathway is at least partially internal to the microphone stand.

17. The apparatus of claim 15, wherein the input oxygen connector connects to an oxygen tube that leads to an oxygen source.

18. The apparatus of claim 15, further comprising an oxygen source housing that is incorporated with or connected to the microphone stand.

19. The apparatus of claim 15, wherein the oxygen providing component is output from a distal end of a boom of the microphone stand.

20. The apparatus of claim 15, wherein the oxygen providing component is output from a distal end of a boom of the microphone stand.
